(19) United States
(12) Patent Application Publication
(10) Pub. No.: US 2003/0149088 A1
(43) Pub. Date: Aug. 7, 2003

(54) HIV treatment

(75) Inventor: James Berger Camden, West Chester, OH (US)

Correspondence Address: HAYNES AND BOONE, LLP, 901 MAIN STREET, SUITE 3100, DALLAS, TX, 75202, US

(73) Assignee: University of Arizona Foundation, Tucson, AZ (US)

(21) Appl. No.: 10/280,100

(22) Filed: Oct. 24, 2002

Related U.S. Application Data
(63) Continuation of application No. 09/703,955, filed on Nov. 1, 2000, now abandoned.

(51) Int. Cl. A61K 31/4184

(52) U.S. Cl. 514/395

(57) ABSTRACT

A pharmaceutical composition that can be used to treat HIV is disclosed. The composition comprising an effective amount of a benzimidazole of the formula:

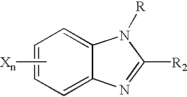

wherein X is hydrogen, halogen, nitro, oxychloro alkyl of less than 7 carbon atoms or alkoxy of less than 7 carbon atoms; n is a positive integer of 4 or less; and R is hydrogen, alkylcarbamoyl wherein the alkyl group has less than 7 carbon atoms, or an alkyl group having from 1 to 8 carbon atoms, and R₂ is NHCOOR, wherein R₁, is an aliphatic hydrocarbon of less than 7 carbon atoms. The pharmaceutically acceptable organic or inorganic addition salts thereof are also used herein. The preferred compounds are methyl-(butylcarbamoyl)-2-benzimidazolecarbamate and 2-methoxycarbonylaminobenzimidazole. In the present invention it has been discovered that the compounds described above are useful for treattment of HIV infection when used alone or in combination with other anti-viral agents.

The present application is a continuation application of U.S. Ser. No. 09/703,955 filed Nov. 1, 2000, incorporated by reference herein, and claims priority to said application under 35 U.S.C. §120.

### TECHNICAL FIELD

This invention is a method of treating HIV with a pharmaceutical composition containing one or more benzimidazole derivatives and/or an adjunct therapy that is another HIV drug.

### BACKGROUND OF THE INVENTION

HIV and other viral infections are one leading cause of death. HIV is a disease in which a virus is replicated in the body which attacks the body's immune system. The HIV virus is not easily destroyed nor is there a good mechanism for keeping the host cells from replicating the virus. A material that targets the HIV virus and inhibits viral replication is highly desirable.

Several drugs have been approved for treatment of this devastating disease, including azidovudine (AZT), didanosine (dideoxyinosine, dd1), d4T, zalcitabine (dideoxycytosine, ddC), nevirapine, lamivudine (epivir, 3TC), saquinavir (Invirase), ritonavir (Norvir), indinavir (Crixivan), and delavirdine (Rescriptor). See M. I. Johnston & D. F. Hoth, Science, 260(5112), 1286-1293 (1993) and D. D. Richman, Science, 272(5270), 1886-1888 (1996 An AIDS vaccine (Salk's vaccine) has been tested and several proteins which are chemokines from CD8 have been discovered to act as HIV suppressors. In addition to the above synthetic nucleoside analogs, proteins, and antibodies, several plants and substances derived from plants have been found to have in vitro anti-HIV activity. However, HIV virus is not easily destroyed nor is there a good mechanism for keeping the host cells from replicating the virus.

Thus, medical professionals continue to search for drugs that can prevent HIV infections, treat HIV carriers to prevent them from progressing to full-blown deadly AIDS, and treat the AIDS patient.

Surprisingly it has been found that certain benzimidazoles that are effective in the treatment of cancer are also effective in the treatment of chronic HIV. Drug screening for HIV virus is performed by testing drugs against the HIV virus incorporated in cancer cell lines. When anticancer drugs are screened in this testing, they are often found to be too toxic. However, if the HIV is grown in a non-cancer cell line, the effectiveness of the drugs is measured. Thus when benzimidazoles of this invention were tested against HIV in non-cancer cells, it was surprisingly found that these benzimidazoles were effective in the treatment of HIV.

### SUMMARY OF THE INVENTION

A method of treating HIV infected animals, and in particular, warm blooded animals and humans, comprising administering a therapeutically effective amount anti-viral compound selected from the group consisting of:

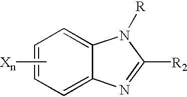

wherein X is hydrogen, nitro, oxychloro, halogen, alkyl of less than 7 carbon atoms or alkoxy of less than 7 carbon atoms; n is a positive integer of 4 or less; and R is hydrogen, an alkyl group of from 1 to 8 carbon atoms, or an alkylcarbamoyl wherein the alkyl group has less than 7 carbon atoms, and R₂ is NHCOOR, wherein R₁ is aliphatic hydrocarbon of less than 7 carbon atoms is claimed. Preferably the benzimidazole is substituted with either a chloro (Cl-) or fluoro in the 5 and 7 positions and the remaining substituents of the benzene ring are hydrogen. Also preferred are tetrafluoro, tetrachloro, and difluoro, dimethyl derivatives of 2-methoxycarbonylaminobenzimidazole.

The most preferred compounds are methyl-(butylcarbamoyl)-2-benzimidazolecarbamate and 2-methoxycarbonylamino-benzimidazole.

The drug can be given daily or from 1 to 4 times a week.

In the present invention it has been discovered that the compounds described above are useful for the inhibition of HIV and the treatment of HIV infection. The present invention also provides methods for the treatment of HIV infection comprising administering to a host infected with HIV a pharmaceutically or therapeutically effective or acceptable amount of a compound as described above along with other HIV drugs.

More specifically, this invention provides an anti-viral composition comprising a pharmaceutical carrier and a benzimidazole derivative as defined herein along with a method for treating viral infections. It is believed that these compositions prevent replication of the HIV virus.

The benzimidazoles described herein can be used in conjunction with other treatments for HIV.

### DETAILED DESCRIPTION OF THE INVENTION

A. Definitions:

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “therapeutically effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. The specific “therapeutically effective amount” will, obviously, vary with such factors as the particular condition being treated, the physical condition of the patient, the type of mammal being treated, the duration of the treatment, the nature of concurrent therapy (if any), and the specific formulations employed and the structure of the compounds or its derivatives. Therapeutically effective amounts are generally recognized as being safe and effective amounts.

As used herein, a “pharmaceutical addition salts” is salt of the anti-viral compound with an organic or inorganic acid. These preferred acid addition salts are chlorides, bromides, sulfates, nitrates, phosphates, sulfonates, formates, tartrates, maleates, malates, citrates, benzoates, salicylates, ascorbates, and the like. Preferably the hydrochloride salt is used.

As used herein, a “pharmaceutical carrier” is a pharmaceutically acceptable solvent, suspending agent or vehicle for delivering the anti-viral agent to the animal or human. The carrier may be liquid or solid and is selected with the planned manner of administration in mind.

As used herein, the “anti-viral compounds” are the benzimidazoles, and their salts. The exact benzimidazoles are described in detail below.

As used herein “viruses” includes viruses which infect animals or mammals, including humans. The term “HIV” encompasses AIDS, retrovirus and related diseases.

As used herein “adjunct therapy” means that the patient in need of the drug is treated or given another drug for the disease in conjunction with the benzimidazole derivatives. This adjunct therapy can be sequential therapy where the patient is treated first with one drug and then the other or the two drugs are given simultaneously.

B. The Anti-Viral Compounds

The anti-viral compounds are benzimidazole derivatives having the following structure:

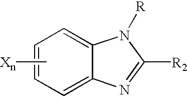

wherein X is hydrogen, halogen, nitro or oxychloro alkyl of less than 7 carbon atoms or alkoxy of less than 7 carbon atoms; n is a positive integer of 4 or less; and R is hydrogen alkylcarbamoyl wherein the alkyl group has 7 carbons or less or an alkyl group of from 1 to 8 carbon atoms and R₂ is NHCOO R₁, wherein R₁ is aliphatic hydrocarbon of less than 7 carbon atoms. Preferably the benzimidazole is substituted with either a chloro or fluoro in the 5 position and the remaining substituents of the benzene ring are hydrogen.

The preferred compounds are methyl-(butylcarbamoyl)-2-benzimidazolecarbamate and 2-methoxycarbonylaminobenzimidazole and the compounds wherein X is chloro, lower alkoxy or hydrogen. The non-toxic, pharmaceutically acceptable acid addition salts with both organic and inorganic acids are also useful herein. Suitable acid addition salts are acid addition salts are selected from the group consisting of chlorides, bromides, sulfates, nitrates, phosphates, sulfonates, formates, tartrates, maleates, malates, citrates, benzoates, salicylates, ascorbates and the like.

The benzimidazole compounds also include prodrugs. “Prodrugs” are considered to be any covalently bonded carriers which release the active parent drug according to the formula of the benzimidazole derivatives described above in vivo when such prodrug is administered to a mammalian subject. Prodrugs of the benzimidazole compounds are prepared by modifying functional groups present in the compounds in such a way that the modifications are cleaved, either in routine manipulation or in vivo, to the parent compounds. Prodrugs include compounds wherein hydroxy or amine groups are bonded to any group that, when administered to a mammalian subject, cleaves to form a free hydroxyl or amino group, respectively. Examples of prodrugs include, but are not limited to, acetate, formate, or benzoate derivatives of alcohol and amine functional groups in the benzimidazole derivatives; phosphate esters, dimethylglycine esters, aminoalkylbenzyl esters, aminoalkyl esters and carboxyalkyl esters of alcohol and phenol functional groups in the benzimidazole derivatives; and the like.

The pharmaceutically acceptable salts of the benzimidazole derivatives include the conventional non-toxic salts or the quaternary ammonium salts of the benzimidazole derivatives formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention are synthesized from the benzimidazole derivatives which contain a basic or acidic moiety by conventional chemical methods. Generally, such salts are prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference. The disclosures of all of the references cited herein are hereby incorporated herein by reference in their entirety.

### SYNTHESIS

The benzimidazole derivatives are prepared in a number of ways well known to one skilled in the art of organic synthesis. The benzimidazole derivatives are synthesized using the methods described below, together with synthetic methods known in the art of synthetic organic chemistry, or variations thereon as appreciated by those skilled in the art. Each of the references cited below are hereby incorporated herein by reference.

These compounds are prepared according to the method described in U.S. Pat. No. 3,738,995 issued to Adams et al, Jun. 12, 1973.

C. Dosage and Dosage Delivery Forms

The type of compound and the carrier and the amount will vary widely depending on the species of animal or human, body weight, and virus or viral infection being treated. The dosage administered will vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired.

The benzimidazole is preferably micronized or powdered so that it is more easily dispersed and solubilized by the body. Processes for grinding or pulverizing drugs are well known in the art. For example, a hammer mill or similar milling device are used. The preferred particle size is less than about 100μ and preferably less than 50μ.

The dosage administered will vary depending upon known factors such as the pharmacodynamic characteristics of the particular active ingredient, and its mode and route of administration; age, sex, health, metabolic rate, absorptive efficiency and/or weight of the recipient; nature and extent of symptoms; kind of concurrent treatment, frequency of treatment; and/or the effect desired.

Dosage forms (compositions) suitable for internal administration contain from about 1.0 milligram to about 5000 milligrams of active ingredient per unit. In these pharmaceutical compositions, the active ingredient will ordinarily be present in an amount of about 0.5-95% by weight based on the total weight of the composition. Based on the body weight of the patient, the dosage may be administered in one or more doses several times per day or per week. Multiple dosage units may be required to achieve a therapeutically effective amount. For example, if the dosage form is 1000 mg, and the patient weighs 40 kg, one pill will provide a dose of 25 mg per kg for that patient. It will provide a dose of only 12.5 mg/kg for a 80 kg patient.

The compounds have shown dose responsiveness in vivo against viruses and cancers in mice at 500 mg/kg, 2500 mg/kg, 3500 mg/kg, 4000 mg/kg, 5000 mg/kg and 6000 mg/kg. Generally a dosage effective in mice translates to about 1/12 of the dosage required in humans. By way of general guidance, for humans a dosage of as little as about 30 milligrams (mg) per kilogram (kg) of body weight and up to about 10000 mg per kg of body weight is suitable. Preferably from 250 mg/kg to about 5000 mg/kg of body weight is used. Most preferably the doses are between 100 mg/kg to about 3000 mg/kg of body weight. However, a dosage of between about 2 milligrams (mg) per kilogram (kg) of body weight to about 400 mg per kg of body weight is also suitable for some indications.

Intravenously, the most preferred doses may range from about 1 to about 1000 mg/kg/minute during a constant rate infusion. Benzimidazole derivatives may be administered in a single daily dose, or the total daily dosage may be administered in divided doses of two, three; or four times daily. The benzimidazole derivatives are given in one or more doses on a daily basis or from one to three times a week.

The benzimidazole derivatives may also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

Generally, the dosage in man is lower than for small warm blooded mammals such as mice. A dosage unit may comprise a single compound or mixtures thereof with other compounds or other viral inhibiting compounds or anti-viral compounds. The dosage unit can also comprise diluents, extenders, carriers and the like. The unit may be in solid or gel form such as pills, tablets, capsules and the like or in liquid form suitable for oral, rectal, topical, intravenous injection or parenteral administration.

The benzimidazole derivatives are typically mixed with a pharmaceutically acceptable carrier. This carrier can be a solid or liquid and the type is generally chosen based on the type of administration being used. The active agent can be coadministered in the form of a tablet or capsule, as an agglomerated powder or in a liquid form. Examples of suitable solid carriers include lactose, sucrose, gelatin and agar. Capsule or tablets are easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms would also include minerals and other materials to make them compatible with the type of injection or delivery system chosen.

When used in an adjunct therapy, the ratio of the benzimidazole of this invention to the adjunct therapy can be from 1:0.001 to about 1:1. The exact therapeutic amount of the adjunct therapy can easily be determined by one skilled in the art. Generally, the amount of the adjunct therapy used will be equal to or less that that used alone for the treatment of HIV. For example, AZT or Zidobudine is used at the generally accepted dosage. Since many of these HIV drugs are used in combination treatments, one skilled in the art can easily determine the exact doses that should be used in combination with the drugs claimed herein.

### EXAMPLES OF FORMULATION

The benzimidazole compounds (active ingredients) of this invention are administered to inhibit virus growth or viral infections by any means that produces contact of the active ingredient with the agent's site of action in the body of a mammal or animal. They can be administered by any conventional means available for use in conjunction with pharmaceuticals, either as individual therapeutic agents or in a combination of therapeutic agents. They can be administered alone, but generally are administered with a pharmaceutical carrier selected on the basis of the chosen route of administration and standard pharmaceutical practice.

The benzimidazole derivatives are administered in oral dosage forms as tablets, capsules (each of which includes sustained release or timed release formulations), pills, powders, granules, elixirs, tinctures, suspensions, syrups, and emulsions. The benzimidazole derivatives may also be administered in intravenous (bolus or infusion), intraperitoneal, subcutaneous, or intramuscular form, all using dosage forms well known to those of ordinary skill in the pharmaceutical arts.

In the methods of the present invention, the compounds herein described in detail can form the active ingredient, and are typically administered in admixture with suitable pharmaceutical diluents, excipients, or carriers (collectively referred to herein as a pharmaceutically acceptable carrier or carrier materials) suitably selected with respect to the intended form of administration consistent with conventional pharmaceutical practices.

For instance, for oral administration in the dosage unit form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. For oral administration in liquid dosage form, the oral drug components are combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The benzimidazole derivatives can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Benzimidazole derivatives may also be coupled with soluble polymers as targetable drug carriers. Such polymers can include polyvinylpyrrolidone, pyran copolymer, polyhydroxylpropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

Useful pharmaceutical dosage forms for administration of the compounds of this invention are illustrated as follows:

Capsules

A large number of unit capsules are prepared by filling standard two-piece hard gelatin capsules each with 100 to 500 milligrams of powdered active ingredient, 5-150 milligrams of lactose, 5-50 milligrams of cellulose, and 6 milligrams magnesium stearate.

Soft Gelatin Capsules

A mixture of active ingredient in a digestible oil such as soybean oil, cottonseed oil or olive oil is prepared and injected by means of a positive displacement pump into gelatin to form soft gelatin capsules containing 100-500 milligrams of the active ingredient. The capsules are washed and dried.

Tablets

A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

Injectable

A parenteral composition suitable for administration by injection is prepared by stirring 1.5% by weight of active ingredient in 10% by volume propylene glycol and water. The solution is made isotonic with sodium chloride and sterilized.

Suspension

An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

The present invention also includes pharmaceutical kits useful, for example, for the treatment of HIV infection, which comprise one or more containers containing a pharmaceutical composition comprising a therapeutically effective amount of a benzimidazole derivative. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. In the present disclosure it should be understood that the specified materials and conditions are important in practicing the invention but that unspecified materials and conditions are not excluded so long as they do not prevent the benefits of the invention from being realized.

D. Adjunct Therapy

One or more benzimidazole derivatives can be combined with other antiviral agents or potentiators. Potentiators are materials that affect the body's response to the anti-viral agent. In the case of HIV an adjunct therapy with AZT, TC-3, protease inhibitors or azidovudine (AZT), didanosine (dideoxyinosine, ddI), d4T, zalcitabine (dideoxycytosine, ddC), nevirapine, lamivudine (epivir, 3TC), saquinavir (Invirase), ritonavir (Norvir), indinavir (Crixivan), and delavirdine (Rescriptor) is effective. The preferred anti-viral agents are AZT, TC-3 or protease inhibitors.

It can also be used in combination with other therapies and other benzimidazoles. For example, thiabendazole or 2-(4-thiazolyl)benzimidazole can be used. Thiabendazole has been shown to be effective in the treatment of HIV. See U.S. Pat. No. 5,880,144 issued Mar. 9, 1999, to Camden, which is incorporated herein by reference. Thiabendazole is sold under the names Mentizole; Omnizole; Thiaben; Thibenzole, Bovizole; Eprofil; and Equizole as an anthelmintic or fungicide. Thiabendazole is prepared according to the method described in Brown et al., J. Am. Chem. Soc., 83,1764 (1961) and Grenda et al., J. Org. Chem., 30, 259 (1965).

In additions the benzimidazole derivatives claimed herein can also be used with N-chlorophenylcarbamates or N-chlorophenylthiocarbamates, preferably with chloropropham. These compounds have the following structure

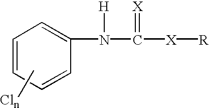

wherein n is from 1 to 3, X is oxygen or sulfur and R is selected from the group consisting of hydrogen, lower alkyl and lower alkenyl, cyclohexyl, phenalkyl of up to 8 carbon atoms and phenyl, and the pharmaceutically acceptable salts of these compounds.

Preferred compounds are those in which R is alkyl with 1 to 4 carbons, preferably, isopropyl and X is oxygen, n is 1 and the chloro group is in the 3 position on the phenyl group. N-3-chlorophenylcarbamate is a most preferred compound. See U.S. Pat. No. 5,629,341 issued May 13, 1997 to Camden for a description of how to treat HIV with these compounds, the disclosure of which is incorporated herein by reference.

These compounds are prepared according to the method described in U.S. Pat. No. 2,695,225 issued to Witman (1954) and U.S. Pat. No. 2,734,911 issued to Strain (1956).

Also certain (5-aryl-1,2,4-thiadazol)-3-yl thioureas can be used in conjunction with the benzimidazoles disclosed herein for the treatment of HIV. These compounds are selected from the group consisting of (5-aryl-1,2,4-thiadiazol)-3-yl thiourea derivative or (5-aryl-1,2,4-thiadiazol)-3-yl urea derivative having the formula:

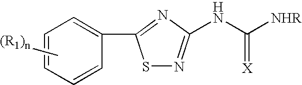

wherein X is oxygen or sulfur, R is hydrogen or alkyl having from 1-3 carbons, n is 0-4, R₁ is independently selected from the group consisting of hydrogen, alkyl having from 1 to 7 carbon atoms, chloro, bromo or fluoro, oxychloro, alkoxy having the formula —O(CH₂)_{y}CH₃ wherein y is from 1 to 6 or a pharmaceutical addition salt or prodrug thereof.

The therapeutic dosage for this drug is from about 1 mg/kg body weight to about 6000 mg/kg body weight.

The preferred compound is:

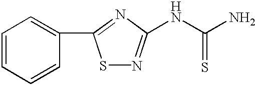

(5-Phenyl-1,2,4-thiadizol)-3-yl thiourea is prepared by the method described in Kurzer, et al, J. Chem. Soc. Perkin Trans. 1(2), 311-314 (1985) and Kurzer, et al., J. Heterocycl. Chem., 26 (2), 355-60 (1989).

The adjunct therapy can be sequential, that is the treatment with one agent first and then a second agent, or it can be treatment with both agents at the same time. The sequential therapy can be within a reasonable time after the completion of the first therapy before beginning the second therapy. The treatment with both agents at the same time can be in the same daily dose or in separate doses. For example, treatment with one agent on day 1 and the other on day 2. The exact regimen will depend on the disease being treated, the severity of the infection and the response to the treatment.

E. Method of Treatment

The method of treatment can be any suitable method that is effective in the treatment of the particular virus type that is being treated. The method of applying an effective amount also varies depending on the virus or viral infection being treated and the severity or stage of infection. It is believed that oral treatment or parenteral treatment by intravenous, subcutaneous, or intramuscular application of the benzimidazole compounds, formulated with an appropriate carrier, additional viral inhibiting compound or compounds or diluent to facilitate application will be the preferred method of administering the compounds to warm blooded animals.

The actual time and dosage will depend on the virus being treated and the desired blood levels.

The following examples are illustrative and are not meant to be limiting to the invention.

The following examples illustrate the effectiveness of methyl -(butylcarbamoyl)-2-benzimidazolecarbamate and 2-methoxycarbonylaminobenzimidazole, 2-(4-thiazolyl)-1H-benzimidazole, against HIV.

### EXAMPLE 1

### HIV Testing

An in vitro screening test of carbendazim against an HIV virus, ROJO in PBMC, available from Southern Research Center is performed using the following assay.

Reverse Transcriptase Inhibition Assay.

A recombinant, purified HIV-1 reverse transcriptase (RT) enzyme provided by Dr. Steven Hughes (ABL,NCI-FCRDC) is used. Characterization of the RT inhibitory properties of selected test compounds is performed utilizing a RT assay described by Boyer et al (1993) with minor modifications. Briefly recombinant RT enzymes are assayed in microtiter plates in a 100 ml reaction mixture containing 25 mM Tris-HCI, pH 8.0, 75 mM KCl, 8 mM MgCl₂, 2 mM DTT, 10 mMdGTP, 0.01 U rC:dG template (Pharmacia), 10 mCi [P³²]-a-dGTP (800 Ci/mmol), and the test compound at indicated concentrations.

The RT enzyme concentration employed in these assays ranged from 0.4-0.9 mgm/ml for the different recombinant proteins; all the RT enzymes reactions are allowed to proceed for 30 min at 37° C. before termination of the enzyme reaction by addition of 10% TCA; 100 mg of heat-denatured, sonicated salmon sperm DNA is also added to aid DNA precipitation and recovery.

Upon termination of the enzyme reaction, the TCA precipitated DNA is harvested onto glass fiber filters (GF/C), washed twice with ice-cold 10% TCA and subjected to liquid scintillation counting. To increase sample throughput and minimize sample handling of this assay, a 96 well glass fiber filter plate and vacuum manifold (Millpore) is used to harvest and wash the DNA.

The labeled DNA samples are subsequently counted directly in the multi-well plate by addition of 20 ml scintillation fluid (OptiPhase Super Mix, Wallac) to each well and using a MicroBeta 96 well scintillation counter (Wallac).

The results of one test is shown below:

### Reverse Transcriptase Activity Carbendazim Against ROJO in PBMC

| | Concentration (μg/ml) | | | | |
|---|---|---|---|---|---|
| | 100 | 32 | 10 | 3.2 | 1 |
| Sample 1 | 132 | 124 | 136 | 200 | 1394 |
| Sample 2 | 84.0 | 180 | 216 | 100 | 685.0 |
| Sample 3 | 152.0 | 88.0 | 108 | 116 | 577.0 |
| Mean | 122.7 | 130.7 | 153.3 | 138.7 | 885.3 |
| % Virus Control | 0.5 | 0.6 | 0.6 | 0.6 | 3.7 |
| | Concentration (μg/ml) | | | | |
| | 0.32 | 0.1 | 0.032 | 0.01 | 0 |
| Sample 1 | 200 | 1394.0 | 23225 | 24174 | 23646 |
| Sample 2 | 8787 | 16747 | 17108 | 32124 | 23646 |
| Sample 3 | 11249 | 12569 | 19000 | 22024 | 23646 |
| Mean | 11143 | 17513 | 22851 | 26107 | 23646 |
| % Virus Control | 47.1 | 74.1 | 96.6 | 110.4 | 100 |

In a similar test of AZT against ROJO in PBMC, the following results were obtained.

### Reverse Transcriptase Activity

| | Concentration μg/ml | | | | |
|---|---|---|---|---|---|
| | 4 | 1.3 | 0.4 | 0.13 | 0.04 |
| Sample 1 | 108 | 68 | 340 | 108 | 176 |
| Sample 2 | 104 | 120 | 80 | 168 | 108 |
| Sample 3 | 84 | 92 | 96 | 56 | 220 |
| Mean | 98.7 | 93.3 | 172 | 110.7 | 168 |
| % virus control | 0.4 | .04 | 0.7 | 0.5 | 0.7 |
| | Concentration μg/ml | | | | |
| | 0.013 | 0.004 | 0.0013 | 0.0004 | 0 |
| Sample 1 | 1.024 | 1.005 | 1.056 | 1.043 | 1.035 |
| Sample 2 | 1.061 | 1.004 | 1.021 | 0.966 | 1.035 |
| Sample 3 | 1.056 | 1.048 | 1.070 | 1.143 | 1.035 |
| Mean | 1.047 | 1.019 | 1.049 | 1.051 | 1.035 |
| % virus control | 101.2 | 98.5 | 101.4 | 101.5 | 100 |

Toxicity Values were determined by the following method:

Toxicity quantification involves the XTT-based evaluation. Assays were designed to characterize the long term effects of the compounds on virus production and to characterize the longer term effects of the compounds on virus production from chronically HIV infected cells.

Cells chronically infected with HIV isolate, for example ROJO in PBMC, are cultured in RPMI1640 tissue culture medium supplemented with 10% fetal bovine serum and antibiotics. Selection is performed by culturing the cells in the presence of the compound to be tested in T25 flasks. Other infected cells with no added drug are used as the control cells. Cells are allowed to grow to a density of approximately 1×106 cells/ml and are then passaged at a 1:10 dilution. After a period of time, usually one week intervals of drug treatment, cells are evaluated to determine if the inhibitory activity of the compound has been affected by treatment of the cells with either compounds. The drug concentration in the flask is then increased two-fold and the cells maintained as above.

The cell populations contain integrated copies of the HIV genome and constitutively produce HIV at relatively high levels or are latently infected and only produce virus after stimulation with phorbol esters, tumor necrosis factor or IL6 (U1 and ACH2). Virus production was reduced. Reductions in virus products were observed when quantifying supernatant reverse transcriptase activity.

### Toxicity Values (Cell titer—O.D. @ 490/650 nm) Carbendazim Against ROJO in PBMC

Concentration μg/ml

| | 100 | 32 | 10 | 3.2 | 1 |
|---|---|---|---|---|---|
| Sample 1 | 0.839 | 0.864 | 1.001 | 1.013 | 1.019 |
| Sample 2 | 0.782 | 0.835 | 0.858 | 1.003 | 1.003 |
| Sample 3 | 0.799 | 0.836 | 0.973 | 0.959 | 0.991 |
| Mean | 0.807 | 0.845 | 0.944 | 0.992 | 1.004 |
| % cell control | 77.9 | 81.6 | 91.2 | 95.8 | 97.0 |

Concentration μg/ml

| | 0.32 | 0.1 | 0.032 | 0.01 | 0 |
|---|---|---|---|---|---|
| Sample 1 | 1.031 | 1.145 | 1.143 | 1.115 | 1.035 |
| Sample 2 | 1.050 | 1.080 | 1.198 | 1.180 | 1.035 |
| Sample 3 | 1.067 | 1.246 | 1.204 | 1.118 | 1.035 |
| Mean | 1.049 | 1.157 | 1.182 | 1.138 | 1.035 |
| % cell control | 101.4 | 111.8 | 114.2 | 109.9 | 100 |

In the same test of AZT against ROJO, the following results are obtained.

### Toxicity Values (Cell titer—O.D. @ 490/650 nm) Carbendazim Against ROJO in PBMC

Concentration μg/ml

| | 4 | 1.3 | 0.4 | 0.13 | 0.04 |
|---|---|---|---|---|---|
| Sample 1 | 0.878 | 0.983 | 1.059 | 1.006 | 0.958 |
| Sample 2 | 0.800 | 0.848 | 1.008 | 0.914 | 1.054 |
| Sample 3 | 1.006 | 1.038 | 10.94 | 0.947 | 1.029 |
| Mean | 0.925 | 0.956 | 1.054 | 0.956 | 1.014 |
| % cell control | 89.3 | 92.4 | 101.8 | 92.3 | 97.9 |

Concentration μg/ml

| | 0.013 | 0.004 | 0.0013 | 0.0004 | 0 |
|---|---|---|---|---|---|
| Sample 1 | 1.024 | 1.005 | 1.056 | 1.043 | 1.035 |
| Sample 2 | 1.061 | 1.004 | 1.021 | 0.966 | 1.035 |
| Sample 3 | 1.056 | 1.048 | 1.070 | 1.143 | 1.035 |
| Mean | 1.047 | 1.019 | 1.049 | 1.051 | 1.035 |
| % cell control | 101.2 | 98.5 | 101.4 | 101.5 | 100 |

Additional Chronic HIV Studies

Chronic HIV-1 infected cells U1 were derived from an acute HIV-1 infection of the promonocytic cell line, U937. The chronic HIV-1 infected cells, ACH-2 were derived from an acute HIV-1 infection of the T cell line, A3.01.

These cells were cultured in medium and the phorbol ester, PMA. PMA causes the cells (both U1 and ACH-2) to be activated and not divide but it also causes the U-1 cells to differentiate. This results in fewer cells in the PMA-treated cultures than the media alone cultures. Cell viability was measured when these cell lines were treated with the test compound.

Both cell lines constituitively produce a small amount of HIV-1. ACH-2 cell lines tend to produce more HIV-1 than U1 cells as shown by p-24 ELISA. When either cell line is cultured in the presence of PMA there is an increase in the quantity of HIV-1 produced as measured by the p-24 antigen ELISA.

In addition, the number of institute positive HIV mRNA expressing cells per microscopic field is measured. Comparisons can be made from these numbers since the same number of cells were adhered to the glass slides for each drug concentration (10×10⁶ cells/ml).

2-(Methoxycarbonylamino)benzimidazole suppressed replication in the HIV monocytes by 9% and the T-cell HIV replication was increased by 44%. The positive control was interferon which suppressed HIV monocytes replication by 80% and suppressed T-cell HIV replication by 60%.

Acute HIV Testing

In an in vitro acute model for HIV 2-(methoxycarbonylamino)benzimidazole inhibited viral replication by 100% at 4 μg/ml and AZT inhibited viral replication by 98% at 1 μg/ml. 2-(4-thiazolyl)-1H-benzimidazole inhibited viral replication by 98% at 60 μg/ml.

The therapeutic index (TI), the ratio of the toxic dose of drug to efficacious dose of drug for 2-(4-thiazolyl)-1H-benzimidazole is 2.8 versus 12, 500 for AZT. The TI for 2-(methoxycarbonylamino)benzimidazole is 1.8.

What is claimed is:

1. A pharmaceutical composition comprising a therapeutically effective amount of: 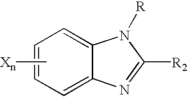
wherein X is hydrogen, halogen, nitro, oxychloro alkyl of less than 7 carbon atoms or alkoxy of less than 7 carbon atoms; n is a positive integer of 4 or less and R is hydrogen, alkylcarbamoyl wherein the alkyl group has less than 7 carbon atoms, or an alkyl group having from 1 to 8 carbon atoms, and R₂ is NHCOOR, wherein R₁ is aliphatic hydrocarbon of less than 7 carbon atoms or the pharmaceutically acceptable inorganic or acid addition salts thereof and a therapeutically effective amount of an adjunct therapy.

2. A pharmaceutical composition according to claim 1 comprising a pharmaceutically acceptable carrier and from about 250 mg to about 5000 mg of a benzimidazole selected from the group consisting of: 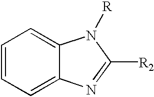
wherein R is hydrogen, alkylcarbamoyl wherein the alkyl group has 1 to 4 carbon atoms, or an alkyl having from 1 to 8 carbon atoms and R₂ is NHCOOR, wherein R₁, is aliphatic hydrocarbon of less than 7 carbon atoms or the pharmaceutically acceptable organic or inorganic acid addition salts thereof.

3. A pharmaceutical composition according to claim 2 wherein said benzimidazole is methyl-(butylcarbamoyl)-2-benzimidazolecarbamate or 2-methoxycarbonyl-aminobenzimidazole.

4. A pharmaceutical composition according to claim 1 wherein said pharmaceutical acceptable acid addition salts are selected from the group consisting of chlorides, bromides, sulfates, nitrates, phosphates, sulfonates, formates, tartrates, maleates, malates, citrates, benzoates, salicylates, ascorbates and mixtures thereof.

5. A pharmaceutical composition according to claim 4 comprising from about 500 mg to about 5000 mg of said benzimidazole.

6. A pharmaceutical composition according to claim 5 wherein said salt is a hydrochloride salt.

7. A pharmaceutical composition according to claim 1 wherein said adjunct therapy comprises a member selected form the group consisting of protease inhibitors, AZT, 3TC, ddC, dd1, thiabendazole, (5-aryl-1,2,4-thiadiazol)-3-yi thiourea, N-chlorophenylcarbamates and N-chlorophenylthiocarbarnates, and mixtures thereof.

8. A pharmaceutical composition according to claim 1 wherein the ratio of benzimidazole to adjunct therapy is from about 1:0.001 to about 1:1.

9. A method of treating HIV comprising administering therapeutically effective amount of a benzimidazole of the formula: 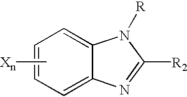
wherein X is hydrogen, halogen, nitro, oxychloro alkyl of less than 7 carbon atoms or alkoxy of less than 7 carbon atoms; n is a positive integer of 4 or less; and R is hydrogen, alkylcarbamoyl wherein the alkyl group has less than 7 carbon atoms, or an alkyl group having from 1 to 8 carbon atoms, and R₂ is NHCOOR, wherein R₁, is aliphatic hydrocarbon of less than 7 carbon atoms or pharmaceutically acceptable salts thereof.

10. A method according to claim 9 comprising from about 100 mg to about 6000 mg of said benzimidazole having the formula: 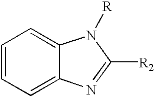
wherein R is hydrogen, alkylcarbamoyl wherein the alkyl group has less than 7 carbon atoms or alkyl group having from 1 to 8 carbon atoms, and R₂ is NHCOOR, wherein R₁, is aliphatic hydrocarbon of less than 7 carbon atoms or the pharmaceutically acceptable salts thereof.

11. A method according to claim 9 wherein said benzimidazole is selected from the group consisting of methyl-(butylcarbamoyl)-2-benzimidazolecarbamate or 2-methoxycarbonyl-aminobenzimidazole.

12. A method according to claim 11 wherein said pharmaceutical acceptable acid addition salts are selected from the group consisting of chlorides, bromides, sulfates, nitrates, phosphates, sulfonates, formates, tartrates, maleates, malates, citrates, benzoates, salicylates, ascorbates and mixtures thereof.

13. A method according to claim 10 wherein said composition is in a liquid form, and wherein said liquid dosage form is selected from the group consisting of aqueous solutions, emulsions, suspension solutions, and suspensions reconstituted from non-effervescent and effervescent preparations.

14. A method according to claim 13 wherein said liquid dosage form further comprises a member selected from the group consisting of suspending agents, diluents, sweeteners, flavorants, colorants, preservatives, emulsifying agents and coloring agents, and mixtures thereof.

15. A method of treating HIV infections comprising administering a therapeutically effective amount of an adjunct therapy in combination with a benzimidazole of the formula: 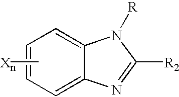
wherein X is hydrogen, nitro, oxychloro halogen, alkyl of less than 7 carbon atoms or alkoxy of less than 7 carbon atoms ; n is a positive integer of 4 or less; and R is hydrogen, alkylcarbamoyl wherein the alkyl group has less than 7 carbon atoms or an alkyl group having from 1 to 8 carbon atoms, and R₂ is NHCOOR₁, wherein R₁, is aliphatic hydrocarbon of less than 7 carbon atoms or the pharmaceutically acceptable organic or inorganic addition salts thereof.

16. A method according to claim 17 wherein said adjunct therapy comprises a member selected from the group consisting of AZT, TC-3, protease inhibitors, thiabendazole, N-chlorophenylcarbamates, N-chlorophenylthiocarbamates or (5-aryl-1,2,4-thiadiazolyl)-thioreas.

17. A method according to claim 16 wherein said benzimidazole is administered in a solid form and wherein said solid form includes a carrier selected from the group consisting of lactose, sucrose, gelatin and agar.

18. A method according to claim 17 wherein from about 1500 mg/kg to about 5000 mg/kg of said benzimidazole is administered.

19. A method according to claim 18 wherein said benzimidazole is administered in a liquid form and wherein said liquid dosage form is selected from the group consisting of aqueous solutions, alcohol solutions, emulsions, suspensions, and suspensions reconstituted from non-effervescent and effervescent preparations and suspensions in pharmaceutically acceptable fats or oils.

20. A method according to claim 16 wherein said adjunct therapy is selected from the group consisting of ACT, TC-3, or protease inhibitors.
